(19) United States
(12) Patent Application Publication
(10) Pub. No.: US 2022/0401233 A1
(43) Pub. Date: Dec. 22, 2022

(54) JOINT FOR AN ORTHOPEDIC DEVICE

(71) Applicant: OTTOBOCK SE & CO. KGAA, Duderstadt (DE)

(72) Inventors: André MÜLLER, Duderstadt (DE); Marcus LÜRSSEN, Göttingen (DE); Markus TÜTTEMANN, Waltrop (DE); David BRAND, Duderstadt (DE); Marie-Luise RÖHRICHT, Erlangen (DE)

(73) Assignee: OTTOBOCK SE & CO. KGAA, Duderstadt (DE)

(21) Appl. No.: 17/787,554

(22) PCT Filed: Dec. 18, 2020
(86) PCT No.: PCT/EP2020/087066

§ 371 Date: Jun. 20, 2022

(30) Foreign Application Priority Data
Dec. 20, 2019 (DE) 10 2019 135 544.3

(51) Int. Cl. A61F 2/60

(52) U.S. Cl. CPC A61F 2/604; A61F 2002/5006

(57) ABSTRACT

A joint for an orthopedic device, the joint comprising: a first element; a spring support mounted to the first element and having at least one spring element; and a second element, the second element being pivotally mounted to the first element in a first swiveling direction counter to a first force applied by the at least one spring element and in an opposite second swiveling direction counter to a second force applied by the at least one spring element.

### CROSS REFERENCE TO RELATED APPLICATIONS

This is a national phase application of International Application No. PCT/EP2020/087066, filed 18 Dec. 2020, which claims the benefit of German Patent Application No. 10 2019 135 544.3, filed 20 Dec. 2019, the disclosures of which are incorporated herein, in their entireties, by this reference.

### TECHNICAL FIELD

The invention relates to a joint for an orthopedic device, the joint comprising a first element, a spring support with at least one spring element mounted to the first element, and a second element.

### BACKGROUND

This type of joint is known from DE 10 2010 014 334 A1 and DE 10 2015 112 283 A1, for example, in the form of an ankle joint. Such ankle joints can be used in leg or lower leg orthoses. Here, it may be practical for therapeutic reasons to restrict the length of the pivot movement, i.e. the maximum possible pivot angle of the second element relative to the first element, and, for example, to provide an end stop in one or both opposite directions. In order to prevent too hard of a strike on the end stops, they are generally designed to be spring-loaded and thus damped. This spring system also ensures that the joint for the orthopedic device can only be pivoted when the force applied by the at least one spring element is overcome. This may also be useful for rehabilitation and training purposes.

The strength of the force applied by the at least one spring element is often and preferably designed to be adjustable by means of a so-called pre-load. DE 10 2017 112 997 discloses a corresponding joint in which both the pre-load and angle of engagement can be adjusted in the mounted state of the joint. The angle of engagement is understood to mean the angle between the first element and the second element from which a further pivoting in a respective pivot direction is only possible against the force applied by the spring element. Prior to this, a so-called free-Wheeling region may be arranged, in which the first element can be pivoted relative to the second element without having to overcome a force applied by the at least one spring element.

Joints known from the prior art have two spring supports, each of which has at least one spring element, with a force transmission element being arranged on the respective spring support, which comes into contact with the second element. When the second element is pivoted further relative to the first element in the respective pivot direction, the force transmission element is moved and thus leads, for example, to a compression of a disc spring stack or a helical spring. A disadvantage, however, is that this type of joint requires a relatively large installation space, which is particularly disadvantageous for ankle elements, since they may be worn inside a shoe. In addition, the use of two spring supports, each with at least one spring element, also means that the construction is heavy, which is also disadvantageous.

### SUMMARY

The invention thus aims to further develop a joint according to the preamble in claim 1 so that it can be made to be light and small.

The invention solves the problem addressed by way of a joint for an orthopedic device according to the preamble of claim 1, characterized in that the second element is pivotally mounted to the first element in a first pivot direction counter to a first force applied by the at least one spring element and in an opposite second pivot direction counter to a second force applied by the at least one spring element. The at least one spring element, which is arranged on a single spring support, consequently applies the forces used in each case in both pivot directions. This makes it possible to save an entire spring support and as such to design the structure with a relatively small required installation space and low dead weight.

Advantageously, the spring support has at least two spring elements, one of which applies the first force and one of which applies the second force. This may refer to compression spring elements, for example. Thus, if the second element is pivoted relative to the first element in the first pivot direction, in this case, for example, one of the spring elements is compressed, which is only necessary against the acting spring force. In this case, this is the first force. When the second spring element pivots in the opposite second pivot direction relative to the first element, the second spring element is compressed, thus applying the second force. By means of different designs of the two spring elements, which are preferably interchangeable, particularly preferably independently interchangeable, the first force and the second force can preferably be adjusted independently of each other. This is useful, for example, if a movement of the joint in one of the two pivot directions is to be more strongly damped than the movement in the opposite other pivot direction.

In a preferred configuration, the two spring elements are arranged one inside the other or one behind the other. The arrangement one inside the other is achieved particularly easily if the two spring elements are, for example, helical springs, helical disc springs or disc spring stacks. All these springs have a cavity in their interior in which the other spring element can be arranged. This further reduces the required installation space. Alternatively or additionally, however, the two spring elements can of course be arranged next to each other or one behind the other in the direction of compression. In particular, helical springs, helical disc springs and disc spring stacks have a longitudinal axis. This is preferably the direction about which the helical winding of the respective screw element coils. It is preferably identical to the compression and expansion direction of the spring element. The at least two spring elements are preferably arranged one behind the other in this direction. The at least two spring elements are preferably aligned in such a way that their two longitudinal directions run parallel to each other and, particularly preferably are identical, i.e. coaxial.

In a preferred embodiment, the at least two spring elements are arranged and configured in such a way that one of the two spring elements is loaded in the tensile direction to apply the respective force and is preferably a tensile spring element, and one of the two spring elements is loaded in the compression direction to apply the respective force and is preferably a compression spring element. Particularly preferably, the two spring elements are compression springs. In particular, these are spring elements that are charged with energy by pressure, in particular compressed in the spatial expansion, and then exert a compression force.

If the second element is pivoted in one of the two pivot directions against the force of one of the at least two spring elements, this spring element is compressed when it is loaded in the compression direction and stretched when it is loaded in the tensile direction.

In a particularly preferred embodiment, there is only a single spring element that is both a compression spring element and a tensile spring element. When the second element is pivoted in the first pivot direction, for example, the spring element is compressed, while when the second element is pivoted in the opposite second pivot direction, it is expanded, i.e. extended. Different first and second forces can also be adjusted here through the careful selection of the respective spring characteristic curve. Alternatively, a single spring element can be used, which is pressurized, i.e. preferably compressed, both when the second element is pivoted in the first direction and when the second element is pivoted in the second direction. However, in this case only one pre-load, one spring strength and thus one force curve can be used for both directions of movement.

Preferably, a pre-load of at least one of the spring elements, preferably all spring elements, can be adjusted. Particularly preferably, this can be done independently of each other. For this purpose, there is preferably at least one adjustment element, particularly preferably at least one adjustment element per spring element. In a particularly preferred embodiment, the respective pre-load can also be adjusted in the mounted state of the joint. To this end, the respective adjustment element must be accessible from the outside, even when the joint is mounted. This has the advantage that the joint, and thus the orthopedic device on which the joint is arranged, does not have to be dismantled for individual adjustment of the damping, so that the adjustment can be carried out particularly easily, for example, by an orthopedic technician or even by the patient themselves.

In a preferred embodiment, the second element can be connected to a force transmission element of the spring support in such a way that tensile forces and compressive forces can be transmitted. If the second element is pivoted relative to the first element in the first pivot direction, compressive forces are transmitted, for example, from the second element to the force transmission element. The force transmission element is coupled with the at least one spring element and compresses or expands the spring element, thereby exerting the first force. The spring element is compressed when it is loaded in the compression direction and stretched when it is loaded in the tensile direction. When the second component is pivoted in the opposite second pivot direction, tensile forces are transmitted from the second element to the force transmission element of the spring support. This also results in a movement of the force transfer element relative to the rest of the spring support, and in particular to the spring element, causing the spring element to compress or expand, thereby applying the second force. If there is only one spring element, it is compressed, i.e. loaded in the compression direction, when the second element is pivoted in the first pivot direction, for example, and stretched, i.e. loaded in the tensile direction, when the second element is pivoted in the second pivot direction. The reverse configuration is of course also possible. Of course, it is also possible to use a single spring element in such a way that it is stretched, i.e. loaded in the tensile direction, or compressed, i.e. loaded in the compression direction, independently of the pivot direction of the second element.

Alternatively, it is of course also possible for the second element to be connected to two force transmission elements of the spring support, which transmit both compressive forces or both tensile forces, for example. In this case, when the second element is pivoted in the first pivot direction, a compressive or tensile force is transmitted via the first force transmission element, while when the second element is pivoted in the opposite second pivot direction, the respective force is transmitted via the second force transmission element.

Such a connection between the second element and the force transmission element of the spring carrier constitutes a separate invention which, in particular, can also be used separately in a joint according to the preamble of claim 1. It is not necessary for the second element to be pivotable in a first pivot direction against a first force and in the opposite second pivot direction against a second force and/or for these two forces to be exerted by one or more spring elements of the same spring support. Rather, such a connection can also be practical when the joint for an orthopedic device comprises a first element, a spring support with at least one spring element mounted to the first element, and a second element. It is also advantageous in such a joint if tensile forces and compressive forces can be transmitted through the connection between the second element and a force transmission element of the spring element.

Advantageously, the at least one spring element comprises a helical spring, a helical disc spring, a stack of disc springs and/or a rubber-elastic element, in particular an elastomer block. If more than one spring element is provided, different types of spring element can be combined with one another or the same spring elements can be used.

The at least one spring element preferably applies the first force to the second element only from a first angle of engagement and/or the second force from a second angle of engagement. For example, with the first force in the first pivot direction, this means that when the second element is pivoted in the first pivot direction, the first force does not have to be overcome until the first angle of engagement is reached. Prior to this, pivoting is possible without an acting force. Similarly, it can be advantageous if the second force is applied when the second element is pivoted in the opposite second pivot direction only after a second angle of engagement is reached.

Preferably, the joint features a hydraulic damping unit by way of which the pivoting of the first element relative to the second element is damped in at least one pivot direction, preferably in both pivot directions. Such a hydraulic damping unit preferably comprises at least one cylinder, in which a piston is located that can be displaced along a longitudinal direction of the cylinder. The cylinder preferably has two cylinder chambers, which are preferably located on two sides of the piston and are fluidically connected to each other. If the position is now moved inside the cylinder, the hydraulic medium inside the hydraulic damping unit is pumped from one of the chambers into the second chamber. In this case, the fluidic connection, for example a channel or a tube, represents a flow resistance which causes the damping. Of course, the hydraulic damping unit may also comprise two cylinders, each of which has one cylinder.

The damping is preferably adjustable. Particularly preferably, the damping can be adjusted separately for both pivot directions. An adjustable damping can be achieved, for example, by arranging a throttle value in the fluidic connection, i.e. for example in the channel or the tube, by means of which the flow resistance caused by the fluidic connection can be adjusted. If the damping can be separately adjusted for both pivot directions, two fluidic connections may be provided between the two chambers, each comprising a throttle valve. Non-return valves positioned in the two fluidic connections ensure that the respective fluidic connection can only be flowed through in one direction.

### BRIEF DESCRIPTION OF THE DRAWINGS

In the following, some examples of embodiments of the present invention will be explained in more detail by way of the attached figures: They show:

FIGS. 1 to 3—a sectional view through a joint according to a first example of an embodiment of the present invention in a range of positions,

FIG. 4—a schematic sectional view through another joint,

FIG. 5—a sectional view through a joint according to a further example of an embodiment,

FIGS. 6-8—schematic sectional views through joints according to further examples of an embodiment of the present invention,

FIGS. 9 to 10—representation of a joint according to another example of an embodiment of the present invention, and

FIGS. 11 to 13—representations of a part of the joint from FIGS. 9 and 10 in various positions.

### DETAILED DESCRIPTION

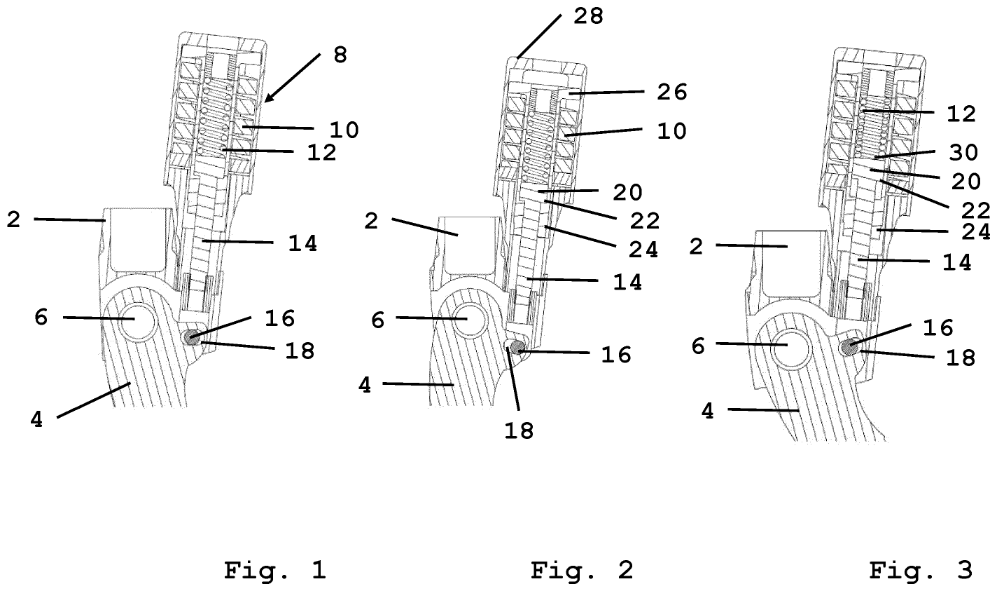

FIG. 1 shows a section view through a joint according to a first example of an embodiment of the present invention that features a first element 2 and a second element 4, which are arranged such that they can be pivoted about a pivot axis 6. A spring support 8 is located on the first component 2, said spring support featuring a first spring element 10 and a second spring element 12. The spring support 8 also has a force transmission element 14, at the lower end of which a pin 16 is located that engages in an elongated hole 18 provided on the second element 4, thereby establishing a connection between the force transmission element 14 and the second element 4, through which both compressive forces and tensile forces can be transmitted.

FIGS. 2 and 3 show the representation from FIG. 1, wherein the joint has now been moved into different positions.

FIG. 2 shows that the second element 4 has been pivoted in the clockwise direction about the pivot axis 6 relative to the first element 2. As a result, a tensile force is applied via the pin 16 in the elongated hole 18 to the force transmission element 14, which then moves downwards. It features a head 20 with a ring-shaped end stop 22. In FIG. 1 and in FIG. 2, the ring-shaped end stop 22 is in contact with a compression component 24, which extends inside the first spring element 10 and at whose upper end in FIG. 2 a compression head 26 is arranged. If the force transmission element 14 moves downwards as a result of the pivot movement of the second element 4 relative to the first element 2, the compression component 24 and the compression head situated on it also move downwards, thereby compressing the first spring element 10 and applying the first force. Both the force transmission element 12 and the compression component with the compression head can be designed as a single piece or multiple pieces in the form of multiple connected components.

Compared to the situation in FIG. 1, it is clear in FIG. 2 that the compression head 26 has performed a downward movement and now forms an intermediate space between the compression head 26 and the upper end of a sleeve 28, which is an outer boundary of the spring support.

FIG. 3 depicts the reverse situation. The second element 4 has been pivoted about the pivot axis 6 in the anti-clockwise direction relative to the first element 2. As a result, a compressive force is applied via the pin 16 in the elongated hole 18 to the force transmission element 14, which is now displaced upwards. It is clear that the end stop 22 of the head 20 of the force transmission element 14 no longer rests on the corresponding compression component 24. Rather, a contact surface 30 of the head 20 has moved the second spring element 12 upwards, thereby compressing it, the second force having been applied.

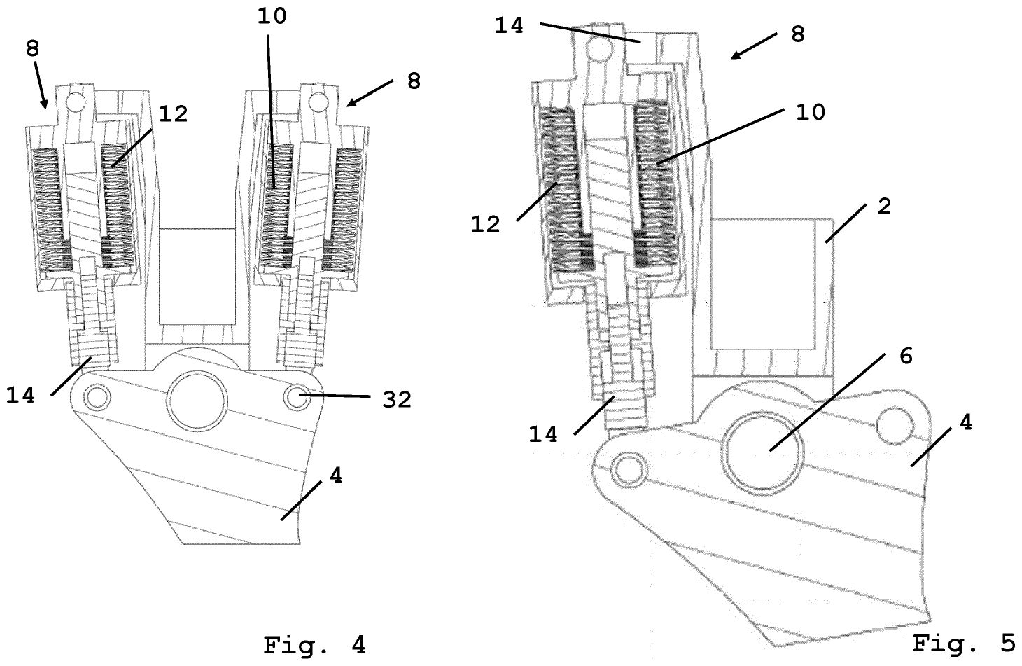

FIG. 4 shows an embodiment of the joint in which two spring supports 8 are arranged on the second element 4. They each support one of the spring elements 10, 12, both of which are designed as compression springs in the example of an embodiment shown. Since the force transmission elements 14 are arranged on the second element 4 in such a way that compressive forces and tensile forces can be transmitted, this embodiment is sufficient. Alternatively, both spring elements 10, 12 could also be designed as tension springs. Of course, it is also possible to used spring supports 8 that have a first spring element 10 and a second spring element 12, which in turn can be designed as tensile elements, compression elements or different elements, i.e. one tensile element and one compression element.

The eyelets 32 are brought into overlap with bores in the second element 4. A peg or bolt is then pushed through these openings, thereby achieving the connection shown in FIGS. 4 and 5.

FIG. 5 depicts how a coupling of the force transmission element 14 to the second element 4 can be achieved for another embodiment of the joint with the first component 2, the second component 4 and the pivot axis 6. The force transmission element 14 is hinged to the second element 4 and is thus moved both clockwise and anti-clockwise when the second element 4 is pivoted relative to the first element 2.

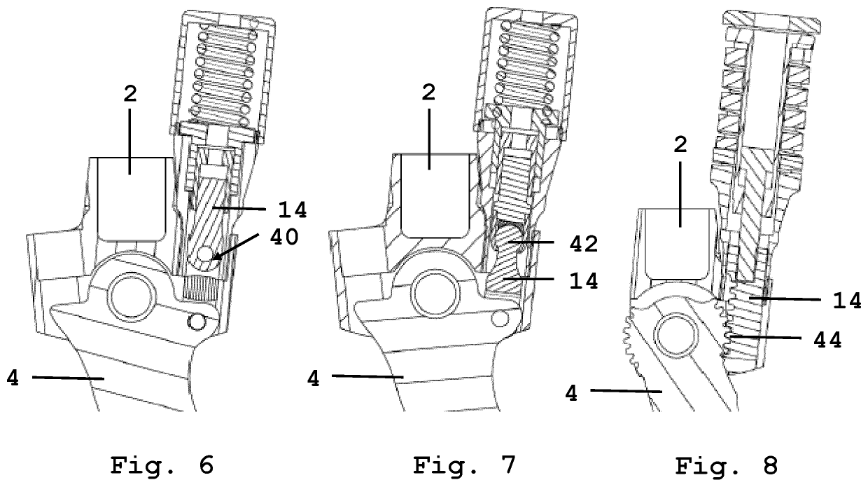

FIGS. 6, 7 and 8 each depict various embodiments of a spring support mounted in a joint with the first component 2 and the second component 4. The representation focuses especially on the attachment of the respective force transmission element 14 to the second element 4. While a connecting rod joint 40 is used in FIG. 6, the embodiment in FIG. 7 has a ball joint 42 and FIG. 8 has a corresponding toothing 44.

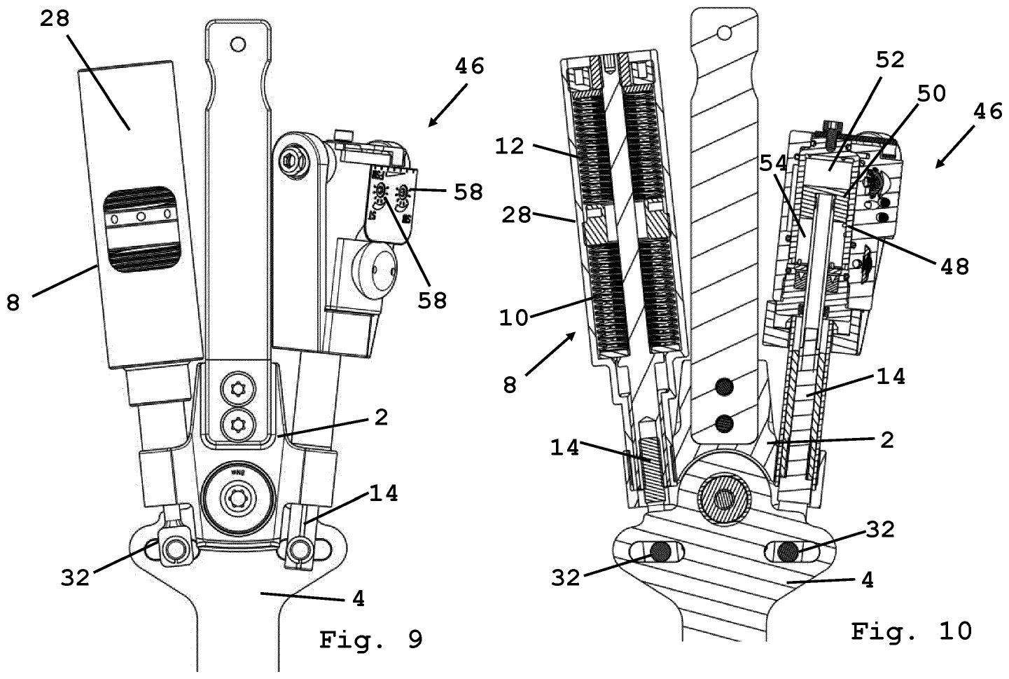

FIG. 9 shows a schematic representation of the joint with the first element 2 and the second element 4. The left-hand area of the joint, depicted in a schematic 3D view in FIG. 9, comprises the spring support 8, where the eyelet 32, which is arranged by means of a pin or peg on an elongated hole of the second element 4, is shown. The sleeve 28 is also depicted, which can contain the spring elements 10, 12 and the other elements and components. A hydraulic damping unit 46 is depicted on the right-hand side of the joint.

FIG. 10 shows a schematic sectional view of the joint from FIG. 9. The first spring element 10 and the second spring element 12 are shown in the sleeve 28. The hydraulic damping unit 46 features a cylinder 48, in which a piston 50 can move, said piston dividing the interior of the cylinder 48 into a first cylinder chamber 52 and a second cylinder chamber 54. On the piston 50 is a piston rod 56, which is connected to a force transmission element 14. Like the force transmission element 14 on the left-hand side of the joint, which constitutes part of the spring support 8, this force transmission element features an eyelet 32 that is attached to a second elongated hole of the second element 4 via a pin or peg.

If the second element 4 is pivoted relative to the first element 2, the force transmission element 14 is also moved, causing the piston 50 to be displaced inside the cylinder 48. As a result, a hydraulic medium is pumped from the first cylinder chamber 52 into the second cylinder chamber 54 or vice-versa. To this end, fluidic connections are provided between the two cylinder chambers 52, 54, which are not shown for the sake of clarity. However, in FIG. 9 one can recognize adjustment devices 58, by way of which throttle valves—not depicted—can be opened or closed, so that a flow resistance generated by the throttle valves in the fluidic connections can be adjusted. As a result, the damping for movements of the two elements 2, 4 relative to each other can be adjusted separately in both pivot directions.

FIGS. 11 to 13 each show a sectional view through the spring support 8 with the sleeve 28 as it is used in the joint according to FIGS. 9 and 10. In the central area is the force transmission element 14, at the end of which the eyelet 32 is located. The force transmission element 14 is attached to a central rod 60, which has a lower compression projection 62 and an upper compression projection 64. An end stop 66 is attached in the central area of the sleeve 28, said end stop preferably being infinitely adjustable. The first spring element 10 extends between the end stop 66 and the lower compression projection 62 and the second spring element 12 is arranged between the end stop 66 and the upper compression projection 64.

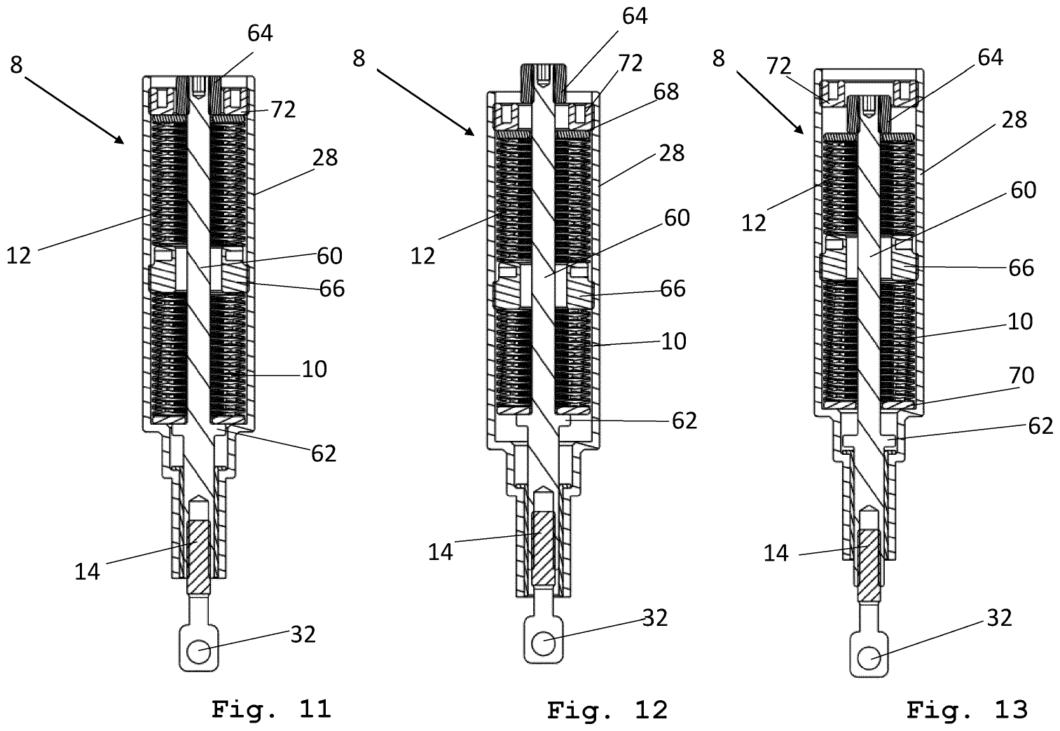

FIG. 11 shows the spring support 8 in a neutral position. Conversely, in FIG. 12 the force transmission element 12 has been displaced upwards along with the central rod 60, i.e. pressure is exerted by the second element 4. As a result, the central rod as well as the upper compression projection 64 attached to it and the lower compression projection 62 are moved upwards. The first spring element 10 is thus compressed between the lower compression projection 62 and the end stop 66, so that a restoring force is exerted. The second spring element 12, on the other hand, remains unchanged and is not pre-loaded, as the distance between the end stop 66 and an upper contact surface 68, on which the second spring element 12 rests, does not change. Only the first spring element 10 is pre-loaded.

FIG. 13 shows the reverse situation, in which a tensile force is applied downwards on the eyelet 32 and therefore on the force transmission element 14 and the central rod 60. The second spring element 12 is now compressed, since the distance between the end stop 66 and the upper compression projection 64 decreases. The first spring element 10, on the other hand, remains unchanged and is not pre-loaded, as the distance between the end stop 66 and a lower contact surface 70 remains unchanged.

The spring element 10 can be pre-loaded by displacing the end stop 66. The spring element 12 can be pre-loaded by displacing an upper end stop 72. Depending on the pre-load of the spring element 12 and the lowering of the upper end stop 72, the upper compression projection 64 may have to be readjusted so that it rests on the contact surface 68. The statics and the starting angle of the ankle joint system can be continuously adjusted via the central rod 60 and the force transmission element 14 due to the threaded connection.

### REFERENCE LIST

2 first element
4 second element
6 pivot axis
8 spring support
10 first spring element
12 second spring element
14 force transmission element
16 pin
18 elongated hole
20 head
22 end stop
24 compression component
26 compression head
28 sleeve
30 contact surface
32 eyelet
34 compression projection
36 positive-locking element
38 opening
40 connecting rod joint
42 ball joint
44 toothing
46 hydraulic damping unit
48 cylinder
50 piston
52 first cylinder chamber
54 second cylinder chamber
56 piston rod
58 adjustment device
60 central rod
62 lower compression projection
64 upper compression projection
66 end stop
68 upper contact surface
70 lower contact surface
72 upper end stop

1. A joint for an orthopedic device, wherein the joint comprises
a first element,
a spring support with at least one spring element mounted on the first element and
a second element,
wherein the second element is pivotally mounted on the first element in a first pivot direction counter to a first force applied by the at least one spring element and in an opposite second pivot direction counter to a second force applied by the at least one spring element.

2. The joint according to claim 1, wherein the spring support has at least two spring elements, one of which applies the first force and one of which applies the second force.

3. The joint according to claim 2, wherein the two spring elements are arranged one inside the other or one behind the other.

4. The joint according to claim 2, wherein the two spring elements are compression springs.

5. The joint according to claim 2, wherein the at least two spring elements are arranged and configured in such a way that one of the two spring elements is loaded in the tensile direction to apply a force and is preferably a tensile spring element, and one of the two spring elements is loaded in the compression direction to apply a force and is preferably a compression spring element.

6. The joint according to claim 2, wherein a pre-load of at least one of the spring elements, preferably all spring elements, is adjustable, preferably independently of each other.

7. The joint according to claim 6, wherein the pre-load can be adjusted in a mounted state of the joint.

8. The joint according to claim 1 wherein the second element is connected to a force transmission element of the spring support in such a way that tensile forces and compression forces can be transmitted.

9. The joint according to claim 1, wherein the at least one spring element comprises a helical spring, a helical disc spring, a stack of disc springs and/or a rubber-elastic element, in particular an elastomer block.

10. The joint according to claim 1, wherein the at least one spring element applies the first force to the second element only from a first angle of engagement and/or the second force from a second angle of engagement.

11. The joint according to claim 1, wherein the joint features a hydraulic damping unit by way of which a pivoting of the first element relative to the second element is damped in at least one pivot direction, preferably in both pivot directions.

12. The joint according to claim 10, wherein the damping is adjustable, preferably separately adjustable for both pivot directions.

13. A joint for an orthopedic device comprising:
a first element and a second element; and
a spring support with at least two adjustable spring elements mounted on the first element;
wherein the second element is pivotally mounted on the first element in a first pivot direction counter to a first force applied by a first of the at least two spring elements and in an opposite second pivot direction counter to a second force applied by a second of the at least two spring elements.

14. The joint according to claim 13, wherein the at least two spring elements are arranged one inside the other or one behind the other.

15. The joint according to claim 13, wherein the at least two spring elements are compression springs.

16. The joint according to claim 13, wherein the at least two adjustable spring elements are arranged and configured in such a way that one of the at least two spring elements is loaded in a tensile direction to apply a force and is preferably a tensile spring element, and one of the two spring elements is loaded in a compression direction to apply a force and is preferably a compression spring element.

17. The joint according to claim 13, wherein each of the at least two adjustable spring elements is independently adjustable.

18. The joint according to claim 13, wherein the a pre-load of each of the at least two adjustable spring elements can be adjusted in a mounted state of the joint.

19. The joint according to claim 12, wherein the second element is connected to a force transmission element of the spring support in such a way that tensile forces and compression forces can be transmitted.

20. A joint for an orthopedic device comprising:
a first element and a second element; and
a spring support with at least two independently adjustable spring elements mounted on the first element;
wherein the second element is pivotally mounted on the first element in a first pivot direction counter to a first force applied by a first of the at least two spring elements and in an opposite second pivot direction counter to a second force applied by a second of the at least two spring elements; and
wherein the at least two independently adjustable spring elements are arranged and configured in such a way that one of the at least two spring elements is loaded in a tensile direction to apply a force and is preferably a tensile spring element, and one of the two spring elements is loaded in a compression direction to apply a force and is preferably a compression spring element.
